(19) United States
(12) Patent Application Publication
(10) Pub. No.: US 2022/0240802 A1
(43) Pub. Date: Aug. 4, 2022

(54) IN-EAR DEVICE FOR BLOOD PRESSURE MONITORING

(71) Applicant: Facebook Technologies, LLC, Menlo Park, CA (US)

(72) Inventors: Morteza Khaleghimeybodi, Bothell, WA (US); Nils Thomas Fritiof Lunner, Redmond, WA (US); Antonio John Miller, Woodinville, WA (US)

(21) Appl. No.: 17/473,845

(22) Filed: Sep. 13, 2021

Related U.S. Application Data
(60) Provisional application No. 63/145,742, filed on Feb. 4, 2021.

(51) Int. Cl. A61B 5/0215; A61B 5/352; A61B 5/00; A61B 5/11

(52) U.S. Cl. CPC A61B 5/02154; A61B 5/352; A61B 5/6817; A61B 2560/04; A61B 5/11; A61B 2562/0204; A61B 2562/0219; A61B 5/6803

(57) ABSTRACT

A system provides blood pressure monitoring for a user. The system includes an in-ear device, a sensor, and a processor. The in-ear device includes in-ear electrodes that capture electrical signals of pulses of a user's heartbeat from within an ear canal of the user. The sensor captures sensor data indicating tissue movements caused by the user's heartbeat. The processor determines a time interval between a peak in an R wave in electrocardiogram (EKG) data generated using the electrical signals and a peak in a waveform representing the tissue movement generated using the sensor data. The processor determines a blood pressure level of the user using the time interval. The sensor may be located on the in-ear device, and may be a motion sensor, an acoustic sensor, or a photoplethysmogram (PPG) sensor.

### CROSS-REFERENCE TO RELATED APPLICATIONS

This application claims the benefit of U.S. Provisional Application No. 63/145,742, filed Feb. 4, 2021, which is incorporated by reference in its entirety.

### FIELD OF THE INVENTION

This disclosure relates generally to blood pressure monitoring, and more specifically to blood pressure monitoring using an in-ear device.

### BACKGROUND

Blood pressure is one of the most critical vital signs, and thus is typically measured and monitored using cuff-based systems to evaluate an individual's health. Blood pressure can vary continuously because of factors such as medication, stress level, physical activity, emotional state, etc. The variance may be within a range of healthy levels. Alternatively, an individual's blood pressure may be lower than healthy levels (hypotension) or higher than healthy levels (hypertension). However, cuff-based monitoring devices are obtrusive when worn by the user. Such devices are typically worn only for short periods of time, such as during a medical examination, and fail to provide continuous monitoring of blood pressure.

### SUMMARY

Embodiments relate to an in-ear device for monitoring the blood pressure level of a user. Some embodiments include a system including an in-ear device, a sensor, and a processor. The in-ear device includes in-ear electrodes configured to capture electrical signals of pulses of a user's heartbeat from within an ear canal of the user. The sensor is configured to capture sensor data indicating tissue movements caused by the user's heartbeat. The processor is configured to determine a time interval between a peak in an R wave in EKG data generated using the electrical signals and a peak in a waveform representing the tissue movement generated using the sensor data. The processor is further configured to determine a blood pressure level of the user using the time interval. In some embodiments, the sensor is located on the in-ear device. The sensor may be a motion sensor, an acoustic sensor, or a photoplethysmogram (PPG) sensor. For example, the sensor may be a motion sensor may capture motion data indicating the tissue movements. In another example, the sensor may be an acoustic sensor may capture audio data indicating the tissue movements. The in-ear device may include one sensor or multiple sensors (e.g., of different type).

Some embodiments include a method for determining blood pressure level of a user. The method includes: capturing, by in-ear electrodes of an in-ear device, electrical signals of pulses of the user's heartbeat from within an ear canal of the user; capturing, by a sensor, sensor data indicating tissue movements caused by the user's heartbeat; determining, by a processor, a time interval between a peak in an R wave in electrocardiogram (EKG) data generated using the electrical signals and a peak in a waveform representing the tissue movement generated using the sensor data; and determining, by the processor, the blood pressure level of the user using the time interval.

Some embodiments include an in-ear device. The in-ear device includes in-ear electrodes, a sensor, and a processor. The in-ear electrodes are configured to capture electrical signals of pulses of a user's heartbeat from within an ear canal of the user. The sensor is configured to capture sensor data indicating tissue movements inside the ear canal of the user caused by the user's heartbeat. The sensor is a motion sensor or an acoustic sensor. The processor is configured to: determine a time interval between a peak in an R wave in electrocardiogram (EKG) data generated using the electrical signals and a peak in a waveform representing the tissue movement generated using the sensor data; and determine a blood pressure level of the user using the time interval.

### BRIEF DESCRIPTION OF THE DRAWINGS

FIG. 1 is a block diagram of a blood pressure monitoring system, in accordance with one or more embodiments.

FIG. 2A is a perspective view of a headset implemented as an eyewear device, in accordance with one or more embodiments.

FIG. 2B is a perspective view of a headset implemented as a head-mounted display, in accordance with one or more embodiments.

FIG. 3 shows a pulse transit time (PTT) analysis using EKG data and sensor data, in accordance with one or more embodiments.

FIG. 4 is a flowchart of a method for determining blood pressure level of a user, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

Blood pressure is generated by an individual's heartbeat and is the force that moves blood through the body. The pulsation of the heart creates biopotentials and causes tissue movements throughout the body, such as the expansion and contraction of the arteries and veins. The time interval between a peak in the biopotential and the tissue movement is related to the blood pressure level of the user. Embodiments relate to an in-ear device for monitoring the blood pressure level of a user based on this relationship. The in-ear device includes in-ear electrodes that capture electrical signals of the pulses of the user's heartbeat from within an ear canal of the user. One or more sensors capture sensor data indicating the tissue movements caused by the user's heartbeat. For example, a motion sensor may capture motion data indicating the tissue movements. In another example, an acoustic sensor may capture audio data indicating the tissue movements. Some or all the sensors may be located on the in-ear device, or elsewhere, such as on a headset and/or wearable device (e.g., watch, bracelet, arm cuff, etc.).

A processor, which may be separate from the in-ear device (e.g., in a headset, cuff, or other device) or in the in-ear device, uses the electrical signals and the sensor data to determine the blood pressure level of the user. For example, the electrical signals are used to generate electrocardiogram (EKG) data including R waves. The sensor data is used to generate a waveform with peaks representing the tissue movements. The processor determines the blood pressure level of the user using a pulse transit time (PTT) analysis. For example, the processor determines a time interval between a peak in an R wave in the EKG data and a peak in the waveform. The processor determines a blood pressure level of the user using the time interval. In general, a longer time-interval corresponds with a lower blood pressure. In some embodiments, blood pressure levels and corresponding time intervals for a user may be determined in a calibration process.

The tissue movement can be captured with different types of sensors. Some sensors that may be used include an acoustic sensor, a motion sensor, or a photoplethysmogram (PPG) sensor. These sensors are small enough to be located on the in-ear device along with the in-ear electrodes while still allowing the in-ear device to fit comfortably in the ear canal of the user. The in-ear device may also provide other functionality, such as audio rendering or hearing assistance. In some embodiments, a sensor for detecting tissue movements caused by heartbeats may be separate from the in-ear device. For example, imaging devices may be attached to a headset or a cuff to capture image data indicating the tissue movements.

As such, the in-ear device can continuously and unobtrusively monitor blood pressure of the user, providing real-time information that can be always on. The in-ear device allows users to monitor their history of blood pressure over time (e.g., across days, months, years, etc.), and facilitates sharing of the blood pressure information with the user or a physician.

Embodiments discussed herein may include or be implemented in conjunction with an artificial reality system. Artificial reality is a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured (e.g., real-world) content. The artificial reality content may include video, audio, haptic feedback, or some combination thereof, any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to create content in an artificial reality and/or are otherwise used in an artificial reality. The artificial reality system that provides the artificial reality content may be implemented on various platforms, including a wearable device (e.g., headset) connected to a host computer system, a standalone wearable device (e.g., headset), a mobile device or computing system, or any other hardware platform capable of providing artificial reality content to one or more viewers.

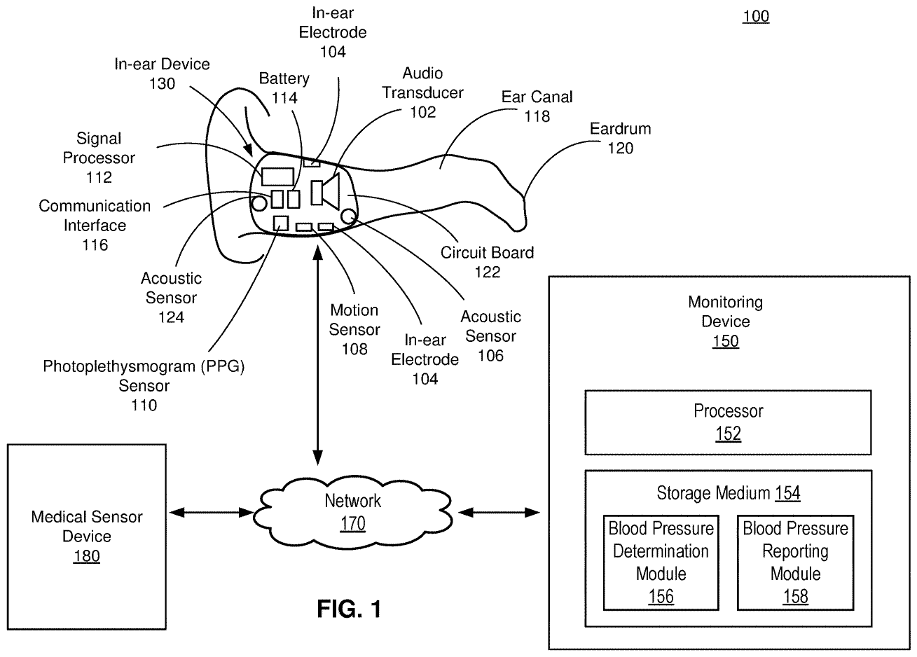

FIG. 1 is a block diagram of a blood pressure monitoring system 100. The blood pressure monitoring system may include an in-ear device 130, a monitoring device 150, a medical sensor device 180, and a network 170. The in-ear device 130 fits within an ear canal 118 of a user near an eardrum 120 and captures various types of data from within the ear canal 118. The monitoring device 150 receives the data from the in-ear device 130 via the network 170 and determines blood pressure levels of the user using the data. Some embodiments of the in-ear device 130 and monitoring device 150 have different components than those described here. Similarly, in some cases, functions can be distributed among the components in a different manner than is described here. For example, some or all of the processing for blood pressure level determination by the monitoring device 150 as described herein may be performed by the in-ear device 130. In some embodiments, some or all the sensors of the in-ear device 130 may also be in the medical sensor device 180.

The in-ear device 130 capture biometric information about the user that can be used to determine blood pressure levels. The in-ear device 130 may include an audio transducer 102, in-ear electrodes 104, an acoustic sensor 106, a motion sensor 108, and a photoplethysmogram (PPG) sensor 110, a signal processor 112, a battery 114, a communication interface 116, and an acoustic sensor 124. These components of the in-ear device 130 may be mounted to a circuit board 122 that connects the components to each other.

The audio transducer 102 is a speaker that generates sound from audio data and outputs the sound into the ear canal 118. The audio transducer 102 may be used to provide audio messages to the user. For example, the audio transducer 102 may be used to communicate blood pressure levels or notify the user when the blood pressure of the user indicates a potential health problem, such as hypotension or hypertension. The audio transducer 102 may also be used to present other types of audio content to the user. In some embodiments, the audio transducer 102 re-broadcasts sound from the local area detected by the acoustic sensor 124, such that the in-ear device 130 provides hear-through functionality even though it is occluding the ear canal 118.

The in-ear electrodes 104 capture electrical signals indicating pulses of the user's heartbeat. The electrical signals represent the biopotentials created by the pulsation of the user's heart. The electrical signals captured by the in-ear electrodes 104 may be used to generate EKG data defining waveform that represents the electrical activity that is taking place within the heart. The EKG may include pulses, each pulse having a P wave, followed by a QRS complex including a Q wave, an R wave, and an S wave, and followed by a T wave. Although two in-ear electrodes 104 are shown, the in-ear device 130 may include additional electrodes to capture the electrical signals.

In some embodiments, the in-ear electrodes 104 are dry electrodes that may be directly in contact with the tissue of the user. A dry electrode does not need gel or some other type of medium or layer between the in-ear electrodes 104 and the tissue. The in-ear electrodes 104 may include hard material electrodes (e.g., including gold-plated brass, iridium oxide, etc.) or soft material electrodes (e.g., including conductive textiles, conductive polymers, carbon allotropes such as graphene or carbon nanotubes, or poly(3,4-ethylenedioxythiophene) polystyrene sulfonate (PEDOT:PSS).

The acoustic sensor 106, motion sensor 108, and PPG sensor 110 are examples of sensors that capture sensor data indicating tissue movements caused by the user's heartbeat. Depending on how the blood pressure is determined, the in-ear device 130 may include one or more of these sensors. The sensor data from a sensor is used to generate a waveform representing the tissue movements over time caused by the user's heartbeat. Each heartbeat of the user results in a pulse in the electrical signals captured by the in-ear electrodes 104 followed by a tissue movement that is captured by a sensor. As discussed in greater detail below, the waveform representing the tissue movement is compared with the EKG data for determination of blood pressure levels.

The acoustic sensor 106 captures audio data of sound pressure inside the ear canal 118 caused by the tissue movements. The in-ear device 130 creates a sealed acoustic chamber within the ear canal 118 when inserted within the ear of the user. The sealed acoustic chamber allows the acoustic sensor to capture the low volume heartbeat-driven acoustic perturbances created in the sealed acoustic chamber by the tissue movements. The audio data captured by the acoustic sensor 106 may be used to generate the waveform representing the tissue movements that is used for determination of blood pressure level.

The acoustic sensor 124 may be an external microphone that is outside of the sealed acoustic chamber when the in-ear device 130 is inserted within the ear of the user. The acoustic sensor 124 captures sound that arrives at the user, and may be used for purposes such as sound cancellation, sound amplification, etc.

The motion sensor 108 captures motion data of the tissue movements inside the ear canal 118 of the user caused by the user's heartbeat. The motion sensor 108 may include a contact microphone, an accelerometer, an inertial measurement unit (IMU), a contact transducer, or some other type of device that captures movement. The motion data captured by the motion sensor 108 may be used to generate the waveform representing the tissue movements that is used for determination of the blood pressure level.

The motion sensor 108 may be placed in contact with the tissue or may be embedded within the in-ear device 130. When embedded, there is no physical contact between the motion sensor 108 and the anatomy of the user. For ease of manufacturing and integration, the motion sensor 108 may be embedded within the in-ear device 130 on the circuit board 122. As the anatomy moves as caused by the heartbeat, the in-ear device 130 also goes through those movements and thus a motion sensor 108 that is embedded within the in-ear device 130 can capture these heartbeat driven motions. When the motion sensor 108 is in contact with the tissue of the user, the motion sensor 108 is placed at the outer surface of the in-ear device 130. The closer the motion sensor 108 is to the veins of the user (e.g., closer to the anterior auricular branches of the superficial temporal artery), the better the signal to noise ratio of the motion data captured by the motion sensor 108.

The photoplethysmogram (PPG) sensor 110 captures optical data of the motion-based pulses of the arteries in the ear canal 118. The PPG sensor 110 may be placed in proximity to the wall of the ear canal 118. The PPG sensor 110 may include a light source and a photodetector. The light source emits light on the tissue which is reflected and absorbed differently based on blood volume in the tissue, and the reflected light is captured by the photodetector. The optical data captured by the PPG sensor 110 may be used to generate the waveform representing the tissue movements that is used for determination of blood pressure level.

To capture these heartbeat-driven intensity variations, the PPG sensor 110 includes at least one light source and one photodetector. In some embodiments, more than one light source and/or more than one photodetector may be used to enhance signal to noise ratio and increase functionality. For example, a combination of three light sources (e.g., having three unique wavelengths) and a photodetector can be used to capture heartbeat-driven intensity variations at multiple wavelengths, thereby enabling heartbeat, breathing rate, and blood oxygen level (SpO2) estimations. In some embodiments, the wavelengths of the three light sources include green (e.g., 532 nm), red (e.g., 632 nm), and infrared (e.g., 830 nm) wavelengths. The signal processor 112 or a controller of the PPG sensor 110 may drive the light sources sequentially so the photodetector can obtain heartbeat driven intensity variation information separately for each wavelength. The signal processor 112 performs various types of processing to facilitate the capturing of sensor data. For example, the signal processor 112 may include an analog to digital converter (ADC) that converts the electrical signals from the in-ear electrodes 104 into EKG data. The ADC may also convert the sensor data from the acoustic sensor 106, motion sensor 108, and PPG sensor 110 into digital data representing waveforms. The signal processor 112 may synchronize the electrical signals or EKG data with the sensor data in time to facilitate determination of blood pressure levels. The signal processor 112 may also include a digital to analog converter (DAC) that converts digital audio data into analog audio data for rendering by the audio transducer 102. For example, the signal processor 112 may provide audio messages indicating that the user's blood pressure is too high or too low to the audio transducer 102 for rendering to the user.

The battery 114 provides power to the other components of the in-ear device 130. The battery 114 allows the in-ear device 130 to operate as a mobile device. The battery 114 may be rechargeable via wire or wirelessly.

The communication interface 116 facilitates (e.g., wireless) connection of the in-ear device 130 to other devices, such as the monitoring device 150 via the network 170. For example, the communication interface 116 may transfer data captured by the sensors of the in-ear device 130 to the monitoring device 150 for determination of blood pressure levels. The in-ear device 130 may also receive blood pressure levels, audio messages, or other types of information determined from the monitoring device 150 via the communication interface 116 for presentation to the user. In some embodiments, the communication interface 116 includes an antenna and a transceiver.

The medical sensor device 180 is a device that includes one or more sensors used to capture sensor data indicating tissue movements of the user. The sensor data captured by the medical sensor device 180 may be used in connection with the electrical signals from the in-ear electrodes 104. The medical sensor device 180 may include one or more of an acoustic sensor 106, motion sensor 108, PPG sensor 110, imaging device, or some combination thereof. The medical sensor device 180 may be a headset, a cuff, a smartphone, a wearable device (e.g., bracelet, watch, etc.), or some other device that can be worn near the skin of the user.

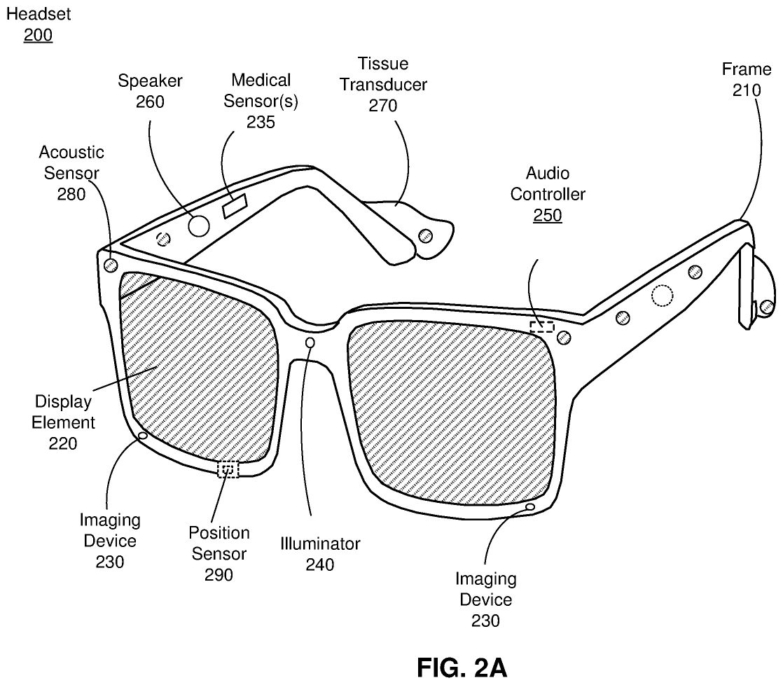

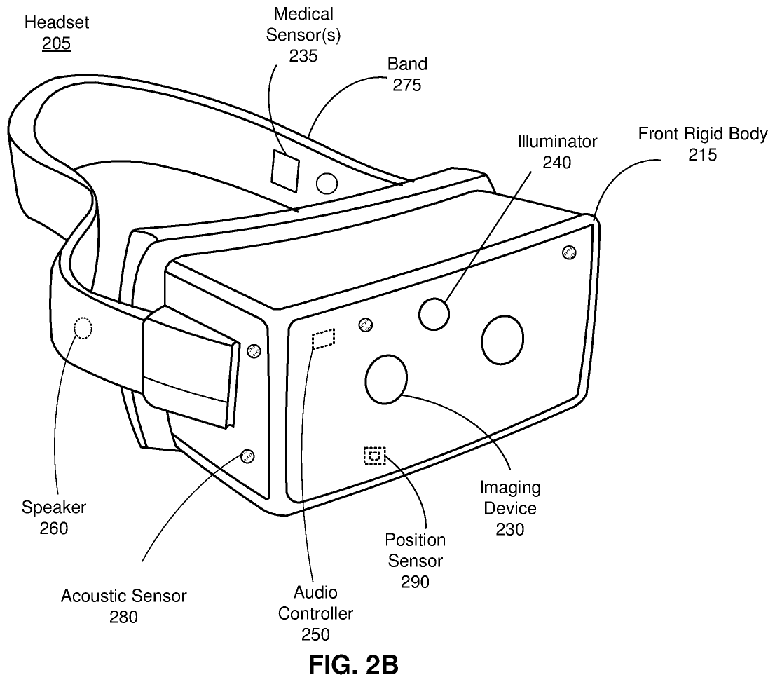

The monitoring device 150 determines the blood pressure level of the user based on the data collected by the in-ear electrodes 104 and other sensors (e.g., from the in-ear device 130 and/or medical sensor device 160). In one embodiment, the monitoring device 150 is a headset or head-mounted display (HIVID), as discussed in greater detail below in connection with FIGS. 2A and 2B. Alternatively, the monitoring device 150 may be a device having computer functionality, such as a desktop computer, a laptop computer, a personal digital assistant (PDA), a mobile telephone, a smartphone, a tablet, an Internet of Things (IoT) device, a virtual conferencing device, a cuff, or another suitable device.

The monitoring device 150 includes a processor 152 and a storage medium 154. The processor 152 operates in conjunction with the storage medium 154 (e.g., a non-transitory computer-readable storage medium) to carry out various functions attributed to the monitoring device 150 described herein. For example, the storage medium 154 may store one or more modules or applications embodied as instructions executable by the processor 152. The instructions, when executed by the processor 152, cause the processor 152 to carry out the functions attributed to the various modules or applications described herein. The processor 152 may be a single processor or a multi-processor system.

The storage medium 154 includes a blood pressure determination module 156 and a blood pressure reporting module 158. The blood pressure determination module 156 performs a PTT analysis to determine blood pressure levels. For example, the blood pressure determination module 106 receives electrical signals and/or EKG data generated by the in-ear electrodes 106 and/or the medical sensor device 180. The blood pressure determination module 106 receives sensor data and/or waveforms representing the tissue movements caused by the user's heartbeat from one or more sensors on the in-ear device 130 (e.g., the acoustic sensor 106, motion sensor 108, PPG sensor 110, or some other type of sensor) and/or on the medical sensor device 180. The blood pressure determination module 156 determines a time interval between a peak in an R wave in the data and a peak in a waveform representing the tissue movement generated using the sensor data. Based on the time interval, the blood pressure determination module 156 determines a blood pressure level of the user. The time-interval between the peak in the R wave and the peak in the waveform representing tissue movements is related to arterial stiffness. The blood pressure determination module 156 may use time-interval to determine the systolic blood pressure (SBP) level and diastolic blood pressure (DBP) level. The time-interval has an inverse relationship to blood pressure levels, with higher time-intervals corresponding with lower blood pressure levels and lower time-intervals corresponding with higher blood pressure levels. The blood pressure determination module 156 determines the blood pressure level based on the inverse relationship and using linear and non-linear regression models. In some embodiments, determining a blood pressure level includes determining a change in blood pressure level, such as an increase or decrease in blood pressure based on a change in the time-interval.

In some embodiments, the blood pressure determination module 156 uses data from different sensors to determine blood pressure levels over time. For example, imaging devices may have a higher power draw than a motion detector or acoustic sensor. The blood pressure determination module 156 may interleave data from different sensors in different ways. For example, one or more low power sensors may be used for a predefined number (e.g., 9) of blood pressure level measurements, and then one or more high power sensors may be used for a predefined number (e.g., 1) of blood pressure level measurements to provide increased accuracy. This process may be repeated to provide low power usage without sacrificing accuracy.

In some embodiments, the blood pressure determination module 156 performs a calibration to relate time-intervals and blood pressure levels for the user. This relationship may vary based on the relative locations of the in-ear electrodes 106 and sensors, as well as biological differences (e.g., gender, age, weight, and BMI, etc.) between different users. During the calibration, blood pressure levels may be measured using a cuff-based system and while electrical signals from the in-ear electrodes 106 and sensor data from one or more of the sensors are collected. The electrical signals and sensor data are used to calculate time intervals, and the time intervals are associated with the measured blood pressure levels (which serves as the ground truth).

In some embodiments, the blood pressure determination module 156 determines blood pressure level using sensor data from an imaging device (e.g., imaging device 230 as shown in FIGS. 2A and 2B), such as a red-green-blue (RGB) camera. The imaging device may be located on the medical sensor device 180 or the monitoring device 150. The imaging device captures image data of small changes in tissue color that indicate the tissue movements of the user. If the imaging device is located on a headset, the imaging device may also be used for facial tracking and/or eye tracking. The blood pressure determination module 156 determines a waveform representing the tissue movements based on the image data, determines a time interval between the peak in the R wave in the EKG data generated using the electrical signals from the in-ear electrodes 106 and a peak in the waveform, and determines a blood pressure level of the user using the time-interval. In some embodiments, the imaging device captures image data of the tissue movements of the user which are used to determine the waveform.

The blood pressure determination module 156 analyzes blood pressure levels to generate messages and reports. For example, a blood pressure level may be compared with threshold levels to determine user health status, such as whether the user's blood pressure level is too high (hypertension) or too low (hypertension). The blood pressure determination module 156 may monitor the history of blood pressure levels over time and generate real-time information regarding blood pressure levels and health status.

The blood pressure reporting module 158 communicates pressure levels, analysis, and reporting to other devices. For example, an audio message may be provided to the in-ear device 130 for rendering by the audio transducer 102. In another example, the blood pressure level, analysis, and reporting may be provided to a display of the monitoring device 150. In another example, the blood pressure reporting module 158 provides the blood pressure level, analysis, and reporting to a device associated with a physician or other healthcare worker. In some embodiments, blood pressure reporting module 158 allows the user to opt in to share the history of the blood pressure levels with their physicians.

Some or all components of the monitoring device 150 may be located in the in-ear device 130. Similarly, some or all the functionality of the monitoring device 150, blood pressure determination module 156, and blood pressure reporting module 158 may be performed by the in-ear device. In some embodiments, the monitoring device 150 is a server connected to the in-ear device 130 via a network 170 that includes the Internet.

The network 170 may include any combination of local area and/or wide area networks, using wired and/or wireless communication systems. In one embodiment, the network 170 uses standard communications technologies and/or protocols. For example, the network 110 includes communication links using technologies such as Ethernet, 802.11 (WiFi), worldwide interoperability for microwave access (WiMAX), 3G, 4G, 5G, code division multiple access (CDMA), digital subscriber line (DSL), BLUETOOTH, Near Field Communication (NFC), Universal Serial Bus (USB), or any combination of protocols. In some embodiments, all or some of the communication links of the network 170 may be encrypted using any suitable technique or techniques.

FIG. 2A is a perspective view of a headset 200 implemented as an eyewear device, in accordance with one or more embodiments. The headset 200 is an example of a monitoring device 150. In some embodiments, the headset 200 is an example of a medical sensor device 180. In some embodiments, the eyewear device is a near eye display (NED). In general, the headset 200 may be worn on the face of a user such that content (e.g., media content) is presented using a display assembly and/or an audio system (e.g., including the in-ear device 130). However, the headset 200 may also be used such that media content is presented to a user in a different manner. Examples of media content presented by the headset 200 include one or more images, video, audio, or some combination thereof. The headset 200 includes a frame, and may include, among other components, a display assembly including one or more display elements 220, a depth camera assembly (DCA), an audio system, and a position sensor 190. While FIG. 2A illustrates the components of the headset 200 in example locations on the headset 200, the components may be located elsewhere on the headset 200, on a peripheral device paired with the headset 200 (e.g., in-ear device 130), or some combination thereof. Similarly, there may be more or fewer components on the headset 200 than what is shown in FIG. 2A.

The frame 210 holds the other components of the headset 200. The frame 210 includes a front part that holds the one or more display elements 220 and end pieces (e.g., temples) to attach to a head of the user. The front part of the frame 210 bridges the top of a nose of the user. The length of the end pieces may be adjustable (e.g., adjustable temple length) to fit different users. The end pieces may also include a portion that curls behind the ear of the user (e.g., temple tip, ear piece).

The frame 210 may include one or more medical sensors 235 that capture tissue movements for blood pressure level determination. The sensor 235 on the headset 200 may work with the in-ear electrodes 104 of the in-ear device 130 to provide the data used to determine blood pressure level. The sensor 235 may include an acoustic sensor 106, a motion sensor 108, or a PPG sensor 110. The sensor 235 are on the frame 210 at a location that is near the skin of the user, such as on the temple of the frame 210. In some embodiments, the sensor 235 may include different sensors from the ones on the in-ear device 130 to allow sensor data of different types to be used in the blood pressure level determination. For example, the in-ear device 130 may include the acoustic sensor 106 while the headset 205 may include the motion sensor 108 or PPG sensor 110.

The one or more display elements 220 provide light to a user wearing the headset 200. As illustrated the headset includes a display element 220 for each eye of a user. In some embodiments, a display element 220 generates image light that is provided to an eyebox of the headset 200. The eyebox is a location in space that an eye of user occupies while wearing the headset 200. For example, a display element 220 may be a waveguide display. A waveguide display includes a light source (e.g., a two-dimensional source, one or more line sources, one or more point sources, etc.) and one or more waveguides. Light from the light source is in-coupled into the one or more waveguides which outputs the light in a manner such that there is pupil replication in an eyebox of the headset 200. In-coupling and/or outcoupling of light from the one or more waveguides may be done using one or more diffraction gratings. In some embodiments, the waveguide display includes a scanning element (e.g., waveguide, mirror, etc.) that scans light from the light source as it is in-coupled into the one or more waveguides. Note that in some embodiments, one or both of the display elements 220 are opaque and do not transmit light from a local area around the headset 200. The local area is the area surrounding the headset 200. For example, the local area may be a room that a user wearing the headset 200 is inside, or the user wearing the headset 200 may be outside and the local area is an outside area. In this context, the headset 200 generates VR content. Alternatively, in some embodiments, one or both of the display elements 220 are at least partially transparent, such that light from the local area may be combined with light from the one or more display elements to produce AR and/or MR content.

In some embodiments, a display element 220 does not generate image light, and instead is a lens that transmits light from the local area to the eyebox. For example, one or both of the display elements 220 may be a lens without correction (non-prescription) or a prescription lens (e.g., single vision, bifocal and trifocal, or progressive) to help correct for defects in a user's eyesight. In some embodiments, the display element 220 may be polarized and/or tinted to protect the user's eyes from the sun.

In some embodiments, the display element 220 may include an additional optics block (not shown). The optics block may include one or more optical elements (e.g., lens, Fresnel lens, etc.) that direct light from the display element 220 to the eyebox. The optics block may, e.g., correct for aberrations in some or all of the image content, magnify some or all of the image, or some combination thereof.

The imaging devices 230 capture image data of the tissue movements of the user for determination of blood pressure levels. An imaging device 230 may be located on the rim of the frame 210 to capture image data of tissue color changes that indicate the tissue movements of the user's cheeks caused by heartbeats. An imaging device may additionally or alternatively be located in other areas, such as on the temple of the frame 210 to capture tissue movements at the side(s) of the user's head.

The DCA determines depth information for a portion of a local area surrounding the headset 200. The DCA includes one or more imaging devices 230 and a DCA controller (not shown in FIG. 2A), and may also include an illuminator 240. In some embodiments, the same imaging devices 230 used for capturing tissue movements are used for determining the depth information. In some embodiments, the illuminator 240 illuminates a portion of the local area with light. The light may be, e.g., structured light (e.g., dot pattern, bars, etc.) in the infrared (IR), IR flash for time-of-flight, etc. In some embodiments, the one or more imaging devices 130 capture images of the portion of the local area that include the light from the illuminator 240. As illustrated, FIG. 2A shows a single illuminator 240 and two imaging devices 230. In alternate embodiments, there is no illuminator 240 and at least two imaging devices 230.

The DCA controller computes depth information for the portion of the local area using the captured images and one or more depth determination techniques. The depth determination technique may be, e.g., direct time-of-flight (ToF) depth sensing, indirect ToF depth sensing, structured light, passive stereo analysis, active stereo analysis (uses texture added to the scene by light from the illuminator 240), some other technique to determine depth of a scene, or some combination thereof.

The audio system provides audio content. The audio system includes a transducer array, a sensor array, and an audio controller 250. However, in other embodiments, the audio system may include different and/or additional components. Similarly, in some cases, functionality described with reference to the components of the audio system can be distributed among the components in a different manner than is described here. For example, some or all of the functions of the controller may be performed by a remote server.

The transducer array presents sound to user. The transducer array includes a plurality of transducers. A transducer may be a speaker 260 or a tissue transducer 270 (e.g., a bone conduction transducer or a cartilage conduction transducer). Although the speakers 260 are shown exterior to the frame 210, the speakers 260 may be enclosed in the frame 210. In some embodiments, instead of individual speakers for each ear, the headset 200 includes a speaker array comprising multiple speakers integrated into the frame 210 to improve directionality of presented audio content. The tissue transducer 270 couples to the head of the user and directly vibrates tissue (e.g., bone or cartilage) of the user to generate sound. The number and/or locations of transducers may be different from what is shown in FIG. 2A. In some embodiments, the transducer is in the in-ear device 130, such as the audio transducer 102. The in-ear device 130 may be a part of the headset 200 or may be separate from the headset 200.

The sensor array detects sounds within the local area of the headset 200. The sensor array includes a plurality of acoustic sensors 280. An acoustic sensor 280 captures sounds emitted from one or more sound sources in the local area (e.g., a room). Each acoustic sensor is configured to detect sound and convert the detected sound into an electronic format (analog or digital). The acoustic sensors 280 may be acoustic wave sensors, microphones, sound transducers, or similar sensors that are suitable for detecting sounds. In some embodiments, the acoustic sensor 280 is a component of the in-ear device 130 and located outside of the ear canal, such as the acoustic sensor 124.

In some embodiments, one or more acoustic sensors 280 may be placed in an ear canal of each ear (e.g., acting as binaural microphones). In some embodiments, the acoustic sensors 280 may be placed on an exterior surface of the headset 200, placed on an interior surface of the headset 200, separate from the headset 200 (e.g., part of some other device), or some combination thereof. The number and/or locations of acoustic sensors 280 may be different from what is shown in FIG. 2A. For example, the number of acoustic detection locations may be increased to increase the amount of audio information collected and the sensitivity and/or accuracy of the information. The acoustic detection locations may be oriented such that the microphone is able to detect sounds in a wide range of directions surrounding the user wearing the headset 200. In some embodiments, an acoustic sensor 280 is a component of the in-ear device 130 located outside the ear canal, such as the acoustic sensor 104.

The audio controller 250 processes information from the sensor array that describes sounds detected by the sensor array. The audio controller 250 may comprise a processor and a computer-readable storage medium. The audio controller 250 may be configured to generate direction of arrival (DOA) estimates, generate acoustic transfer functions (e.g., array transfer functions and/or head-related transfer functions), track the location of sound sources, form beams in the direction of sound sources, classify sound sources, generate sound filters for the speakers 260, or some combination thereof. In some embodiments, the audio controller 250 performs the functionality discussed herein for the processor 152, such as blood pressure level determination.

The position sensor 290 generates one or more measurement signals in response to motion of the headset 200. The position sensor 290 may be located on a portion of the frame 210 of the headset 200. The position sensor 290 may include an inertial measurement unit (IMU). Examples of position sensor 290 include: one or more accelerometers, one or more gyroscopes, one or more magnetometers, another suitable type of sensor that detects motion, a type of sensor used for error correction of the IMU, or some combination thereof.

In some embodiments, the headset 200 may provide for simultaneous localization and mapping (SLAM) for a position of the headset 200 and updating of a model of the local area. For example, the headset 200 may include a passive camera assembly (PCA) that generates color image data. The PCA may include one or more RGB cameras that capture images of some or all of the local area. In some embodiments, some or all of the imaging devices 230 of the DCA may also function as the PCA. The images captured by the PCA and the depth information determined by the DCA may be used to determine parameters of the local area, generate a model of the local area, update a model of the local area, or some combination thereof. Furthermore, the position sensor 290 tracks the position (e.g., location and pose) of the headset 200 within the room. Additional details regarding the components of the headset 200 are discussed below in connection with FIG. 5.

FIG. 2B is a perspective view of a headset 205 implemented as an HIVID, in accordance with one or more embodiments. In embodiments that describe an AR system and/or a MR system, portions of a front side of the HIVID are at least partially transparent in the visible band (˜380 nm to 750 nm), and portions of the HIVID that are between the front side of the HMD and an eye of the user are at least partially transparent (e.g., a partially transparent electronic display). The HMD includes a front rigid body 215 and a band 275. The headset 205 includes many of the same components described above with reference to FIG. 2A, but modified to integrate with the HMD form factor. For example, the HIVID includes a display assembly, a DCA, an audio system, a position sensor 290, and one or more medical sensors 235. FIG. 2B shows the illuminator 240, a plurality of the speakers 260, a plurality of the imaging devices 230, a plurality of acoustic sensors 280, and the position sensor 290. The speakers 260 may be located in various locations, such as coupled to the band 275 (as shown), coupled to front rigid body 215, or may be configured to be inserted within the ear canal of a user (e.g., with in-ear device 130).

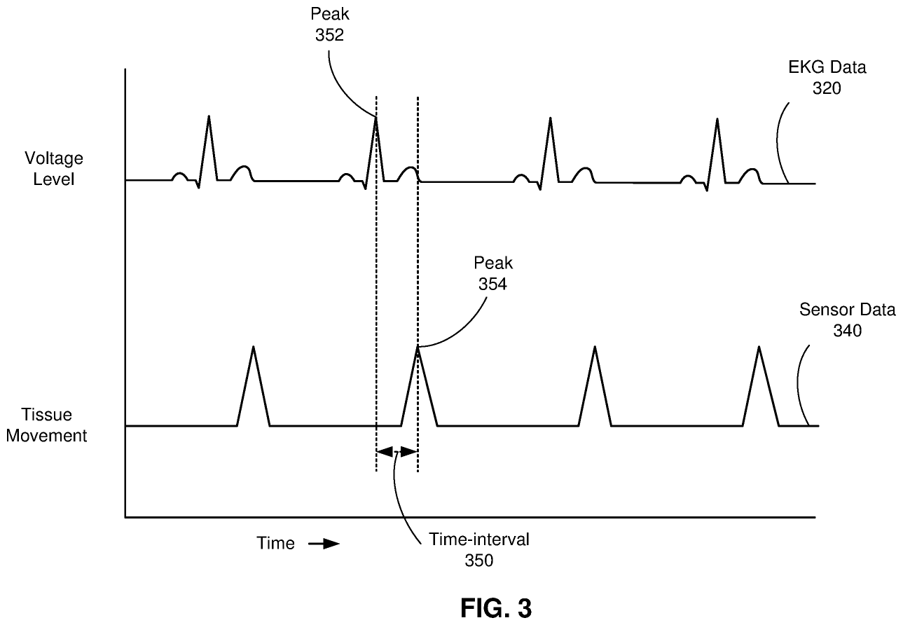

FIG. 3 shows a PTT analysis using EKG data and sensor data, in accordance with one or more embodiments. EKG data 320 is generated from electrical signals of in-ear electrodes 106 and synchronized in time with sensor data 340 generated from a sensor, such as acoustic sensor 106, motion sensor 108, PPG sensor 110, or imaging device 230. In the EKG data 320, each heartbeat corresponds with a pulse including the P wave, followed by the QRS complex including the Q wave, the R wave, and the S wave, and followed by the T wave. A peak 352 of the R wave is shown for one of the pulses. In the sensor data 340, each heartbeat results in a subsequent tissue movement represented by a waveform including pulses. When synchronized in time, each heartbeat corresponds with a pulse in the EKG data 320 followed by a pulse in the waveform of the sensor data 340. A peak 354 is shown for one of the pulses that corresponds with the same heartbeat as the peak 352 of the EKG data. A time interval 350 between the peak 352 and the peak 354 is inversely related to the blood pressure level of the user.

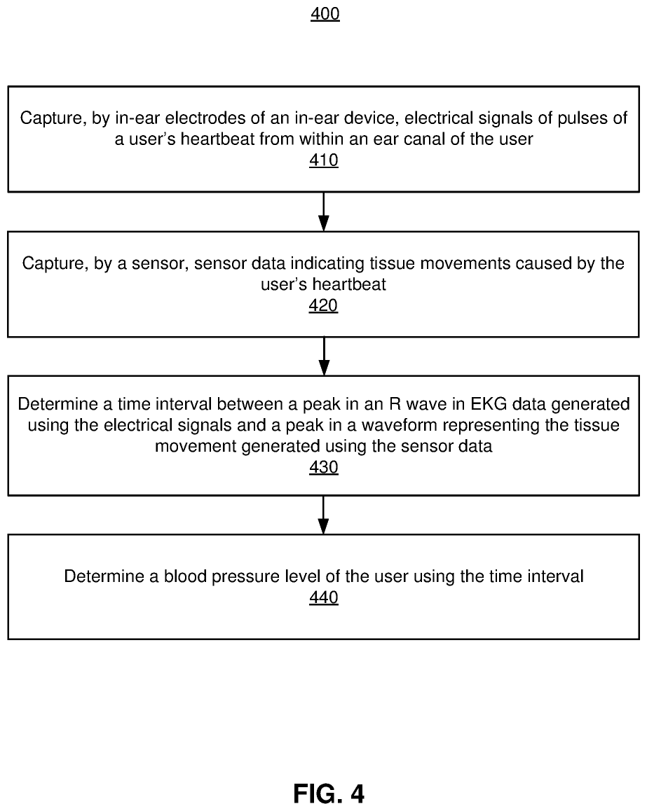

FIG. 4 is a flowchart of a method 400 for determining blood pressure level of a user, in accordance with one or more embodiments. The process shown in FIG. 4 may be performed by components of a blood pressure monitoring system (e.g., blood pressure monitoring system 100). Other entities may perform some or all of the steps in FIG. 4 in other embodiments. Embodiments may include different and/or additional steps, or perform the steps in different orders.

In-ear electrodes 106 of an in-ear device 130 capture 410 electrical signals of pulses of a user's heartbeat from within an ear canal of the user. The electrical signals may be used to generate EKG data including a peak in an R wave for each heartbeat pulse. The EKG data may be generated by the in-ear device 130 or by another device, such as the monitoring device 150. In some embodiments, some or all the EKG data may be captured by another device, such as the medical sensor device 180.

One or more sensors capture 420 sensor data indicating tissue movements caused by the user's heartbeat. The sensor may be located on the in-ear device 130, a medical sensor device 180, a monitoring device 150, or some combination thereof. The one or more sensors may include, e.g., one or more motion sensors (e.g., the motion sensor 108), one or more acoustic sensors (e.g., the acoustic sensor 106), one or more PPG sensors (e.g., the PPG sensor 110), one or more imagine devices (e.g., the imaging device 230), or some combination thereof. The sensor data is synchronized in time with the EKG data to facilitate blood pressure level determination.

A processor 152 determines 430 a time interval between a peak in an R wave in EKG data generated using the electrical signals and a peak in a waveform representing the tissue movement generated using the sensor data. For example, the processor 152 synchronizes the EKG and the waveform in time, identifies a peak in an R wave of the EKG data, identifies a corresponding peak in the waveform, and determines the time interval between the identified peaks.

The processor 152 determines 440 a blood pressure level of the user using the time interval. For example, associations between blood pressure levels and time intervals may be stored in a database or other storage medium. The processor 152 references the database using the determined time interval to determine the blood pressure level of the user. In some embodiments, the processor 152 performs a calibration to relate different time intervals with different blood pressure levels for the user.

The method 400 may be repeated to continuously monitor the blood pressure level of the user over time. In some embodiments, multiple types of sensors may be used to capture different sets of sensor data indicating the tissue movements caused by the user's heartbeat. Different blood pressure levels may be calculated from different sets of sensor data, and a result for the blood pressure level may be selected from one of the results. In another example, the results using different types of sensors are combined (e.g., averaged) to determine a combined result for the blood pressure level.

### Additional Configuration Information

The foregoing description of the embodiments has been presented for illustration; it is not intended to be exhaustive or to limit the patent rights to the precise forms disclosed. Persons skilled in the relevant art can appreciate that many modifications and variations are possible considering the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A system, comprising:
an in-ear device including in-ear electrodes configured to capture electrical signals of pulses of a user's heartbeat from within an ear canal of the user; and
a sensor configured to capture sensor data indicating tissue movements caused by the user's heartbeat; and
a processor configured to:
determine a time interval between a peak in an R wave in electrocardiogram (EKG) data generated using the electrical signals and a peak in a waveform representing the tissue movement generated using the sensor data; and
determine a blood pressure level of the user using the time interval.

2. The system of claim 1, wherein the sensor is located on the in-ear device.

3. The system of claim 2, wherein:
the sensor data includes motion data; and
the sensor is a motion sensor configured to capture the motion data of the tissue movements inside the ear canal of the user.

4. The system of claim 3, wherein the motion sensor is one of:
a contact microphone;
an accelerometer;
an inertial measurement unit; or
a contact transducer.

5. The system of claim 2, wherein:
the sensor data includes audio data; and
the sensor is an acoustic sensor configured to capture the audio data of sound pressure inside the ear canal of the user caused by the tissue movements.

6. The system of claim 5, wherein:
the in-ear device creates a sealed acoustic chamber within the ear canal when inserted within an ear of the user; and
the acoustic sensor is located within the sealed acoustic chamber.

7. The system of claim 5, wherein:
the system further includes a motion sensor configured to capture motion data of the tissue movements inside the ear canal of the user; and
the processor is configured to:
determine a second time interval between the peak in the R wave in the EKG data and a peak in a second waveform representing the tissue movement generated using the motion data; and
determine the blood pressure level using the time interval and the second time interval.

8. The system of claim 2, wherein:
the sensor data includes optical data; and
the sensor is a photoplethysmogram (PPG) sensor configured to capture the optical data of light reflections indicating the tissue movements inside the ear canal of the user.

9. The system of claim 1, wherein the processor is located in a headset.

10. The system of claim 1, wherein the sensor is located on the headset.

11. The system of claim 10, wherein:
the sensor data includes image data; and
the sensor is an imaging device configured to capture the image data indicating the tissue movements.

12. The system of claim 1, wherein the in-ear device further includes a signal processor configured to synchronize the electrical signals and the sensor data and provide the electrical signals and the sensor data to the processor via a wireless connection.

13. The system of claim 1, wherein the processor is configured to perform a calibration to relate different time intervals with different blood pressure levels.

14. A method, comprising:
capturing, by in-ear electrodes of an in-ear device, electrical signals of pulses of a user's heartbeat from within an ear canal of the user;
capturing, by a sensor, sensor data indicating tissue movements caused by the user's heartbeat;
determining, by a processor, a time interval between a peak in an R wave in electrocardiogram (EKG) data generated using the electrical signals and a peak in a waveform representing the tissue movement generated using the sensor data; and
determining, by the processor, a blood pressure level of the user using the time interval.

15. The method of claim 14, wherein the sensor is located on the in-ear device.

16. The method of claim 15, wherein:
the sensor data includes motion data; and
the sensor is a motion sensor configured to capture the motion data of the tissue movements inside the ear canal of the user.

17. The system of claim 15, wherein:
the sensor data includes audio data; and
the sensor is an acoustic sensor configured to capture the audio data of sound pressure inside the ear canal of the user caused by the tissue movements.

18. The system of claim 15, wherein:
the sensor data includes optical data; and
the sensor is a photoplethysmogram (PPG) sensor configured to capture the optical data of light reflections indicating the tissue movements inside the ear canal of the user.

19. The system of claim 14, wherein:
the sensor is located on a headset.
the sensor data includes image data; and
the sensor is an imaging device configured to capture the image data indicating the tissue movements.

20. An in-ear device, comprising:
in-ear electrodes configured to capture electrical signals of pulses of a user's heartbeat from within an ear canal of the user;
a sensor configured to capture sensor data indicating tissue movements inside the ear canal of the user caused by the user's heartbeat, wherein the sensor is one of:
a motion sensor; or
an acoustic sensor; and
a processor configured to:
determine a time interval between a peak in an R wave in electrocardiogram (EKG) data generated using the electrical signals and a peak in a waveform representing the tissue movement generated using the sensor data; and
determine a blood pressure level of the user using the time interval.
